(19) United States
(12) Patent Application Publication
(10) Pub. No.: US 2011/0229535 A1
(43) Pub. Date: Sep. 22, 2011

(54) COMPOSITION HAVING A SIRTUIN ACTIVATOR

(75) Inventors: Monika BEYER, Nidderau-Ostheim (DE); Dirk Teichmüller, Linsengericht (DE); Sarah Teichmüller, Linsengericht (DE); Christian Munk, Steinau-Marborn (DE)

(73) Assignee: ROVI Cosmetics International GmbH, Schluchtern (DE)

(21) Appl. No.: 12/833,165

(22) Filed: Jul. 9, 2010

(30) Foreign Application Priority Data
Mar. 17, 2010 (DE) 10 2010 002 969.6

(51) Int. Cl. A61K 8/14; A61K 9/127; A61K 31/4406; A61K 8/49; A61Q 19/08; A61Q 19/00

(52) U.S. Cl. 424/401; 424/450; 514/355

(57) ABSTRACT

The present invention concerns a composition having at least one sirtuin activator and a carrier system for the at least one sirtuin activator, wherein the carrier system includes lipid vesicles with one or more lipid membranes. To provide an improved action of sirtuin activators in living skin cells, it is proposed in accordance with the invention that the at least one sirtuin activator is contained in the lipid vesicles and the lipid vesicles have a positive surface charge, wherein the positive surface charge is afforded by the lipid vesicles in the lipid membrane or membranes in addition to the lipids from which the vesicles are made up having positively charged molecules as charge generators, wherein said charge generators are selected from quaternary ammonium compound

The present invention concerns compositions having at least one sirtuin activator and a carrier system for the at least one sirtuin activator, wherein the carrier system includes lipid vesicles having one or more lipid membranes, and an active substance composition containing sirtuin activators, the use of such compositions and active substance compositions and cosmetic and/or pharmaceutical formulations containing such compositions or active substance compositions.

The family of sirtuins which are also referred to as SIRT enzymes (Silent Information Regulator) belongs to the NAD(+) dependent histone deacetylases. They regulate important biological processes such as cell ageing or cell longevity, apoptosis, cell differentiation and cell metabolism in general. It was demonstrated that increased activity of sirtuins which is triggered for example by nutritional deficiency exerts a life-prolonging action on cells and delays the ageing process and stress-induced programmed cell death.

It was further found that such influences on the life of cells, besides food shortages, are also exerted by certain active substances, sirtuin activators. The literature describes numerous sirtuin activators of that kind, inter alia resveratrol and derivatives thereof, which increase the activity of sirtuins.

A difficulty in the use of such sirtuin activators is that they must reach the target enzymes, the sirtuins, and should there deploy an activity which is as good as possible, that is to say as great an increase as possible in the activity of the sirtuins. In that respect, particularly in the reduction of skin ageing, it is necessary for the sirtuin activators to pass through the horny layers of the epidermis.

A further problem in increasing the longevity of skin cells is that of activating the specific sirtuins present in the skin cells.

Therefore the object of the present invention is to provide an improved action of sirtuin activators in living skin cells.

According to the invention that object is attained by a composition of the kind set forth in the opening part of this specification, in which the at least one sirtuin activator is contained in the lipid vesicles and the lipid vesicles have a positive surface charge, wherein the positive surface charge is afforded by the lipid vesicles in the lipid membrane or membranes in addition to the lipids from which the vesicles are made up having positively charged molecules as charge generators, wherein said charge generators are selected from quaternary ammonium compounds.

The term quaternary ammonium compounds is used here to denote organic compounds in which a quaternary amino group is contained, wherein in the quaternary amino group four valencies of the nitrogen atom are bonded and the nitrogen atom has a positive charge.

The sirtuin activators are disposed either in the vesicle interior and/or in the vesicle membrane. In certain embodiments one or more sirtuin activators are disposed in the vesicle membrane and one or more other sirtuin activators in the vesicle interior.

It has been found that the use of a carrier system with lipid vesicles into which the sirtuin activators are introduced, wherein the lipid vesicles have quaternary ammonium compounds as a charge carrier, markedly increases the availability and thus the effectiveness of sirtuin activators in cells, in particular skin cells.

The improved availability of the applied active substance is based inter alia on the positive charge of the vesicle surface. The positive charge of the vesicle surface leads to improved adhesion of the vesicles to the surface of cells, in particular living skin cells. That means that the sirtuin activator contained in the vesicles is made available in specifically targeted fashion in living skin cells.

That means that the amount of sirtuin activator or activators which is provided in the skin cells is significantly increased and therewith also the effectiveness thereof is improved, in which respect however those findings are not intended to be binding in relation to the present invention and are also not intended to restrict the scope of the invention.

Particularly preferably the quaternary ammonium compounds are selected from alkyl trimonium salt of the following formula:

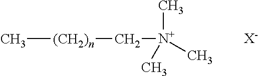

wherein
n is a number of between 18 and 28, and
X⁻ is an inorganic or organic anion.

Such compounds have proven to be particularly suitable for the transport of sirtuin activators in skin cells. The vesicles positively charged by those charge carriers permit particularly good penetration of the sirtuin activators into skin cells and thus targeted provision of the active substances.

Preferably the proportion of sirtuin activator or activators in the present invention, in relation to the total composition, is between 0.0001 and 20% by weight, preferably between 0.0005 and 15% by weight, particularly preferably between 0.001 and 10% by weight.

Preferably the at least one sirtuin activator is selected from the group comprising flavones, stilbenenes, flavonones, isoflavones, catechins, chalcones, tannins, anthocyanides and derivatives thereof. Preferred sirtuin activators are quercitin, piceatannol, resveratrol, isonicotinamide (also known as isoniacinamide), butein, isoliquiritgenin, fisetin, luteolin and 3,6,3′,4′-tetrahydroxyflavone. In addition in certain embodiments the sirtuin activators include cis- or trans-isomers of the substances or a combination of both isomers. Particularly preferred sirtuin activators are selected from quercitin, piceatannol, resveratrol and isonicotinamide.

In a preferred embodiment of the invention at least two sirtuin activators are embraced by the composition. In that respect ‘at least two’ means that precisely two, three, four, five or more sirtuin activators can be included in the composition. The presence of a plurality of sirtuin activators permits a co-operation of the activators. Preferably the at least two sirtuin activators are selected from the group consisting of quercitin, piceatannol, resveratrol and isonicotinamide.

Compositions which include the sirtuin activators resveratrol and isonicotinamide are particularly preferred. That combination of sirtuin activators has proven to be particularly suitable for the activation of sirtuins. In that case the sirtuin activators have a synergistic action in comparison with other active substance combinations. It is assumed that this is based on the different points of attack of the two active substances, either at a given sirtuin and/or at different sirtuins. The invention however is not intended to be bound to that theory and the scope of the invention is not intended to be restricted by that theory.

Preferably the proportion of the sirtuin activators in relation to a composition containing resveratrol and isonicotinamide, in relation to the total composition, is between 0.001 and 1% by weight resveratrol and between 0.1 and 10% by weight isonicotinamide. Particularly good activation is observed in those ranges of quantities.

Preferably n in the above-specified alkyl trimonium salt formula is equal to 22. Particularly preferably the alkyl trimonium salt is behentrimonium chloride. The lipid vesicles which are rendered positive by the behentrimonium chloride permit targeted transport of the sirtuin activators into the cell.

Preferably X⁻ is a halide ion or the anion of an organic acid which is selected from a cosmetically or pharmaceutically compatible carboxylic acid or sulfonic acid. Particularly preferably X⁻ is bromide, chloride, fluoride, iodide, saccharinate, tosylate or methosulfate.

Preferably the positively charged quaternary ammonium compounds are used in an amount of between 0.01 and 10% by weight, preferably between 0.01 and 2.0% by weight, with respect to the total composition.

Preferably the lipid vesicles have a zeta potential in the range of between 1 and 150 mV. The term ‘zeta potential’ describes the electrical potential of a shearing layer of a moved particle in a suspension. Measurement of the zeta potential can be effected by moving particles through an applied electrical field. The zeta potential can then be calculated from the resulting speed of the particles.

In given embodiments the lipid vesicles have a zeta potential in the range of between 30 and 100 mV. In the ease of preferred lipid vesicles the zeta potential is between 40 and 60 mV.

The particle size of the lipid vesicles according to the invention is preferably between 10 and 1000 nm, between 100 and 400 nm, more preferably between 100 and 350 nm, most preferably between 100 and 250 nm.

The composition according to the invention can include vesicles with a lipid membrane (nanosomes), two lipid membranes (liposomes) or a plurality of lipid membranes.

The lipids are preferably selected from ceramides, phospholipids, glycosphingolipids and/or diacylglycosides. They also include sphingomyelines, galactocerebrosides and glucocerebrosides, dihexosides, tri- and tetrahexosides as well as gangliosides. The phospholipids which can be used in the composition for the formation of the vesicles can be selected from all pharmaceutically or cosmetically compatible phospholipids which are capable of forming vesicles (nanosomes or liposomes) in an aqueous medium. Preferred phospholipids are lecithin, phosphatidyl choline, phosphatidyl ethanolamine, and phosphatidyl serine. Particularly preferred is lecithin with a high proportion of phosphatidyl choline. In special embodiments it is also possible to use mixtures of the aforementioned lipids.

In an embodiment the proportion of the lipid or lipids in relation to the total composition is preferably between 1 and 35% by weight, preferably between 3 and 20% by weight, particularly preferably between 5 and 11% by weight.

Also in accordance with the invention are active substance compositions which include at least two sirtuin activators, wherein the at least two sirtuin activators are selected from the group consisting of quercitin, piceatannol, resveratrol and isonicotinamide. Preferably the two sirtuin activators include the compounds resveratrol and isonicotinamide. It is precisely when increasing the longevity of skin cells and the reduction related thereto in skin ageing that the combination of resveratrol and isonicotinamide has proven to be particularly suitable. In that respect those sirtuin activators have a synergistic effect in comparison with other active substance compositions. It is assumed that this is based on the different points of attack of the two active substances, either at a given sirtuin and/or at different sirtuins. The invention however is not intended to be bound to that theory and the scope of the invention is also not intended to be limited by that theory.

In a preferred embodiment the ratio of resveratrol to isonicotinamide in the active substance composition is between 0.001 and 10 to between 10 and 1.

In an embodiment the active substance composition has a carrier system suitable for sirtuin activators, the carrier system being selected from neutral and positively charged lipid vesicles. Positively charged lipid vesicles are preferred. The carrier system of the active substance composition provides that particularly with positively charged lipid vesicles the amount of sirtuin activators provided in the skin cells is significantly increased, which ultimately leads to increased effectiveness of the active substance composition.

The positive charge on the vesicle surface can be achieved in various ways. In an embodiment of the active substance composition the or at least some of the lipid membrane-forming lipids impart a positive charge to the vesicle surface. In a preferred embodiment of the active substance composition in addition to the lipids from which the vesicles are made up the lipid vesicles include positively charged molecules as charge generators.

In a given embodiment the positively charged molecules in the lipid vesicles of the active substance composition correspond to the quaternary ammonium compounds of the composition according to the invention.

In given embodiments the composition or active substance composition additionally includes one or more further active substances. In that respect the further active substances do not belong to the group of the sirtuin activators and preferably include vitamins, glucosides, anti-oxidants and anti-ageing active substances. Such further active substances increase the survival capability of the cells, slow down cell ageing and/or protect the cells from damage. Preferred further active substances are tocopherol (vitamin E) or glyceryl-glycosides. In certain embodiments a tocopherol is included in an amount of between 0.1 and 3% by weight and/or a glyceryl-glucoside is included in an amount of between 0.1 and 10% by weight, with respect to the total composition.

According to the invention the composition or the active substance composition is used for the production of a formulation suitable for the treatment of skin ageing and symptoms associated therewith and for increasing the longevity of skin cells. The formulations include cosmetic and/or pharmaceutical formulations, wherein pharmaceutical formulations are those which are covered by the law governing the manufacture and prescription of drugs. In that respect it is possible to add to the composition which includes positively charged lipid vesicles and at least one sirtuin activator, further active substances which are contained either in the lipid vesicles or outside same and in the rest of the substance or carrier matrix of the formulation. The same applies to the active substance composition.

The formulations can include all adjuvant and additive substances which are usually employed in relation to cosmetic or pharmaceutical preparations. In particular the term ‘adjuvant substance’ in connection with the present invention embraces such additive substances which act on the physical properties of the active substances and/or vesicles and the stability thereof and/or serve for preservation of the composition or active substance composition. Examples of such adjuvant substances are oils, alcohols, polyols, gel-forming agents, buffers, preserving agents, bactericides and germ inhibitors, complexing agents, thickeners or consistency additives.

Preferably the composition according to the invention, active substance compositions and formulations are used for cosmetic and/or pharmaceutical formulations which are suited to topical application. The composition and active substance composition according to the invention can be present in all formulations suitable for topical application, for example in the form of a gel, a cream, an ointment, a spray or a lotion. For that purpose the composition according to the invention or the active substance composition according to the invention can be incorporated into a carrier matrix. The carrier matrix may involve gel formulations, cream formulations, lotions, mask applications and so forth.

For uses in the skin area, the composition according to the invention or the active substance composition according to the invention is preferably used in a lotion, a cream, an ointment, a gel, an aqueous fluid, a face lotion or a mask.

It will be appreciated that all components of the compositions, active substance compositions and formulations according to the invention involve pharmaceutically, cosmetically or dermatologically compatible substances. In accordance with this invention a substance is pharmaceutically, cosmetically or dermatologically compatible if it is non-toxic and can be topically applied in the case of the majority of potential users without the user spontaneously or after a while suffering from an unwanted physiological reaction such as for example reddening or the occurrence of itching.

For the purposes of the original disclosure it is pointed out that all features as can be seen by a man skilled in the art from the present description and the claims, even if they are described in specific terms only in connection with certain other features, can be combined both individually and also in any combinations with others of the features or groups of features disclosed here insofar as that has not been expressly excluded or chemical, physical-chemical, cosmetic, pharmacological or dermatological aspects make such combinations impossible or meaningless. A comprehensive explicit representation of all conceivable combinations of features is dispensed with here only for the sake of brevity and readability of the description.

In addition it is pointed out that it is self-evident to the man skilled in the art that the example hereinafter only serves to set out by way of example a possible embodiment of the present invention. The man skilled in the art will therefore readily understand that in addition all other embodiments having the features or combinations of features according to the invention as recited in the claims lie within the scope of the invention. A comprehensive explicit representation of all conceivable embodiments within the claimed invention is dispensed with here only for the sake of brevity and readability of the description.

### EXAMPLE

An example according to the invention concerns a pharmaceutical formulation of the following composition:

### Phase A:

12.00% by weight of ethanol
3.00% by weight of behentrimonium chloride
7.00% by weight of lecithin
0.60% by weight of resveratrol
1.00% by weight of tocopherol

### Phase B:

0.50% by weight of isoniacinamide
74.35% by weight of water
1.00% by weight of glyceryl-glucoside

### Phase C:

0.50% by weight of potassium phosphate
0.05% by weight of sodium hydroxide

To produce the vesicles in accordance with the example the constituents of phase A were dissolved in ethanol and the constituents of phase B were dissolved in water. Then phase B was added to phase A and homogenised under high pressure. Phase C was added to finish the composition.

The vesicles formed by the lecithin have a particle size of between 100 and 300 nm. The resveratrol is contained in the vesicle membrane while the isoniacinamide is present in the vesicle interior and encapsulated by the lipid layer.

1. A composition having at least one sirtuin activator and a carrier system for the at least one sirtuin activator, wherein the carrier system includes lipid vesicles with one or more lipid membranes, wherein the at least one sirtuin activator is contained in the lipid vesicles and the lipid vesicles have a positive surface charge, wherein the positive surface charge is afforded by the lipid vesicles in the lipid membrane or membranes in addition to the lipids from which the vesicles are made up having positively charged molecules as charge generators, wherein said charge generators are selected from quaternary ammonium compounds.

2. A composition as set forth in claim 1 wherein the charge generators are selected from alkyl trimonium salts of the formula 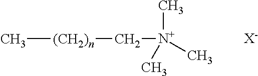
wherein
n is a number of between 18 and 28, and
X⁻ is an inorganic or organic anion.

3. A composition as set forth in claim 1 wherein the proportion of the at least one sirtuin activator in relation to the overall composition is between 0.0001 and 20% by weight.

4. A composition as set forth in claim 1 wherein the at least one sirtuin activator is selected from the group comprising flavones, stilbenenes, flavonones, isoflavones, catechins, chalcones, tannins, anthocyanides and derivatives thereof.

5. A composition as set forth in claim 1 wherein the composition includes at least two sirtuin activators, wherein the at least two sirtuin activators are selected from the group consisting of quercitin, piceatannol, resveratrol and isonicotinamide.

6. A composition as set forth in claim 5 wherein the at least two sirtuin activators include resveratrol and isonicotinamide.

7. A composition as set forth in claim 1 wherein the proportion of the charge generators with respect to the overall composition is between 0.01 and 10% by weight.

8. A composition as set forth in claim 2 wherein X⁻ is bromide, chloride, fluoride, iodide, saccharinate, tosylate or methosulfate.

9. A composition as set forth in claim 1 wherein the zeta potential of the lipid vesicles is between 1 and 150 mV.

10. A composition as set forth in claim 1 wherein the lipid vesicles have a particle size of between 10 and 1000 nm.

11. An active substance composition including at least two sirtuin activators, wherein the at least two sirtuin activators include resveratrol and isonicotinamide.

12. An active substance composition as set forth in claim 11 including a carrier system suitable for sirtuin activators and selected from neutral lipid vesicles and positively charged lipid vesicles.

13. A composition as set forth in claim 1, additionally including one or more further active substances.

14. Use of a composition as set forth in claim 1 for the production of a formulation for the treatment of skin ageing and symptoms associated with skin ageing and for increasing the longevity of skin cells.

15. Cosmetic and/or pharmaceutical formulation for topical application, including a composition as set forth in claim 1.

16. An active substance composition as set forth in claim 11, wherein it additionally including one or more further active substances.

17. Use of an active substance composition as set forth in claim 11 for the production of a formulation for the treatment of skin ageing and symptoms associated with skin ageing and for increasing the longevity of skin cells.

18. Cosmetic and/or pharmaceutical formulation for topical application, including an active substance composition as set forth in claim 11.
